(19) United States
(12) Patent Application Publication
(10) Pub. No.: US 2002/0075450 A1
(43) Pub. Date: Jun. 20, 2002

(54) Compound eye imaging system, imaging device, and electronic equipment

(76) Inventors: Michiharu Aratani, Kanagawa (JP); Tsunefumu Tanaka, Kanagawa (JP); Takeshi Akiyama, Tochigi (JP); Yukihide Kato, Kanagawa (JP)

Correspondence Address: FITZPATRICK CELLA HARPER & SCINTO, 30 ROCKEFELLER PLAZA, NEW YORK, NY, 10112, US

(21) Appl. No.: 09/989,179

(22) Filed: Nov. 21, 2001

(30) Foreign Application Priority Data
Nov. 30, 2000 (JP) 366180/2000 (PAT.

(51) Int. Cl. A61B 3/14

(52) U.S. Cl. 351/206

(57) ABSTRACT

The invention makes easy acquisition of depth distance information of a subject possible by a small-sized device. In a compound eye imaging system comprising a compound eye optical system having a plurality of optical blocks and an imaging element for picking-up object images formed by the optical blocks in imaging ranges provided for each optical block, the optical axes of the optical blocks are made to intersect each other at the object side.

### BACKGROUND OF THE PRIOR ART

1. Field of the Invention

The present invention relates to a compound eye imaging system, more specifically, a compound eye imaging system suitable for imaging devices and electronic equipment such as video cameras, digital still cameras, endoscopes, on-board cameras, computer-attachable cameras, image display telephones, and surveillance cameras.

2. Prior Art

Downsizing and excellent portability have been demanded for the abovementioned imaging devices and electronic equipment. Particularly, downsizing has been in greater demand for imaging systems to be mounted to notebook computers and portable devices.

In a conventional general imaging system, an optical system is comprised by combining a plurality of optical lenses so as to suppress the occurrence of aberrations in the optical system and satisfy desired optical performance.

In such an optical system, to achieve downsizing, reduction in image size and reduction in the diameter of the optical system are considered. However, it is difficult to reduce the image size while maintaining the resolution.

On the other hand, an example which realizes a small-sized optical system by dividing the optical system into a plurality is proposed in Japanese Unexamined Patent Publication No. H10-145802.

In the optical system proposed in the abovementioned publication, the optical system is comprised of a lens array composed of a plurality of lenslets, whereby the lenslets are reduced in size and focal length, and downsizing of the optical system is achieved.

On the other hand, not only picking-up an image of a subject, but also measuring a distance between a camera and a subject (depth distance information) has been demanded. For example, in surveillance camera systems and video teleconference systems, a difference between a distance to a subject person and a distance to the background is used for recognition of the person.

As a method for measuring a depth distance to a subject, for example, there is a method disclosed in Japanese Unexamined Patent Publication No. H10-221066.

However, in the optical system proposed in Japanese Unexamined Patent Publication No. H10-145802, information on the depth distance to a subject cannot be acquired although the optical system is small in size.

In the method proposed in Japanese Unexamined Patent Publication No. H10-221066, a plurality of cameras are used for acquiring information on the depth distance, so that the whole size of the system becomes larger.

Furthermore, in Japanese Unexamined Patent Publications No. H11-122544 and H10-107975, constructions in which a plurality of imaging elements are used for picking-up a subject image are proposed, however, both constructions are not suitable for measuring the subject depth distance.

### SUMMARY OF THE INVENTION

In order to solve the abovementioned problems, the invention provides a compound eye imaging system comprising a plurality of optical blocks and an imaging element for picking-up object images formed by the optical blocks in imaging ranges provided for each optical block, wherein the optical axes of the optical blocks intersect each other at the object side.

Thereby, at least a pair of object images (so called parallactic images) can be obtained through the optical blocks, and it becomes possible to measure the distance (depth distance) to the object based on the positional relationship of these object images on the imaging element and the focal length of the optical blocks.

Furthermore, a construction may be employed in which a drive means is provided which relatively moves the plurality of optical blocks and imaging element to change the space between the optical blocks and imaging element, whereby images of objects at varied distances are formed on the imaging element.

Furthermore, optical action surfaces comprising at least one of the plurality of optical blocks may be formed into an aspherical shape, rotational asymmetric aspherical shape, or diffraction action surface, whereby it becomes possible to satisfactorily correct aberrations. Especially, a diffraction action surface is effective to correct chromatic aberrations.

To obtain a normal object image, images of an object that are more distant than the intersection of the optical axes of the optical block are formed on the imaging element, and the images of each part of the object are synthesized.

### BRIEF DESCRIPTION OF THE DRAWINGS

FIG. 1 is a drawing showing a construction of a compound eye imaging system of a first embodiment of the invention;

FIG. 2 is a drawing showing constructions of a lens array and an imaging element comprising the compound eye imaging system;

FIG. 3 is a sectional view of the compound eye imaging system;

FIG. 4 is an explanatory view of a method for acquiring depth information using the compound eye imaging system;

FIG. 5 is a perspective view of a card type camera of a second embodiment of the invention; and

FIG. 6 is a perspective view of a computer of a third embodiment of the invention.

### DETAILED DESCRIPTION OF THE PREFERRED EMBODIMENT

(First Embodiment)

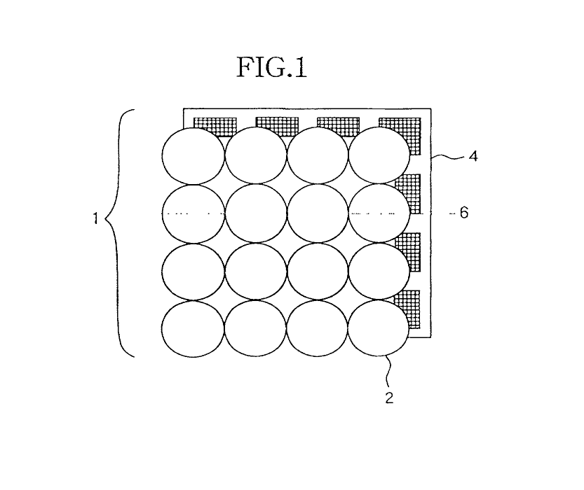

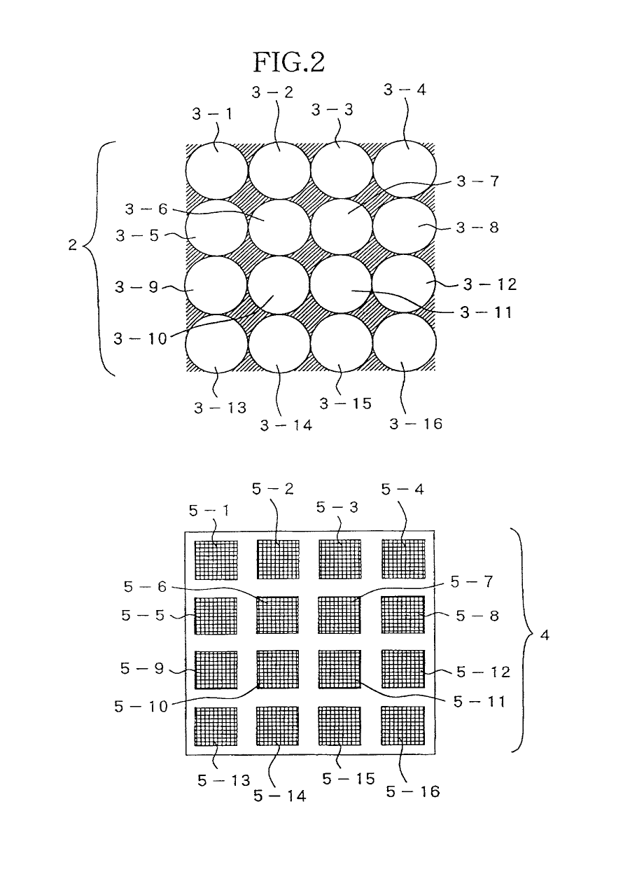

FIG. 1 through FIG. 3 show a construction of a compound eye imaging system of a first embodiment of the invention. This compound eye imaging system can be mounted to imaging devices and electronic equipment such as video cameras, digital still cameras, endoscopes, on-board cameras, computer-attachable cameras, image display telephones and surveillance cameras.

In these figures, compound eye imaging system 1 is comprised of lens array (compound eye optical system) 2 and imaging element 4 comprised of a CCD, CMOS sensor, or the like.

The lens array 2 is comprised of optical blocks 3-1 through 3-16 (in this embodiment, sixteen arranged by four each vertically and horizontally), and at the imaging element 4, the same number of imaging blocks 5-1 through 5-16 as that of optical blocks are formed for each optical block.

The optical blocks 3 are formed from a transparent material such as glass or plastic, and sixteen optical blocks 3 are all connected to each other and unified. The same number of image detecting elements (pixels) as that of the imaging blocks are arranged over the same area for each imaging block.

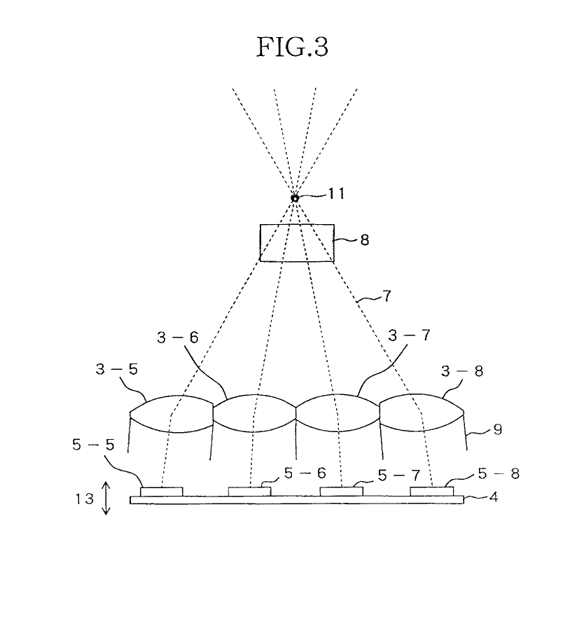

Next, image forming action in the compound eye imaging system 1 is explained with reference to FIG. 3. In FIG. 3 (sectional view along the dotted line 6 of FIG. 1), the dotted lines extending from the optical blocks indicate optical axes of the optical blocks. In this embodiment, the optical axes of the optical blocks are defined by lines passing through the centers of the optical blocks and the imaging blocks. The optical axes are not always straight, and may bend due to refractive action of the optical blocks.

The optical axes of the optical blocks incline from each other so that the interval between the optical axes of one optical block and another optical block becomes narrow at the subject (object) side, and the optical axes of the optical blocks roughly intersect at one point of intersection 11.

For example, among the light rays from the object 8, a light ray that has been made incident on the optical block 3-5 undergoes refractive action from the optical block 3-5 and the image thereof is formed on the imaging block 5-5. Also, the image of a light ray that has been made incident on the optical block 3-6 is formed on the imaging block 5-6. Hereinafter, images of light rays that have been made incident on the optical blocks are formed on the corresponding imaging blocks in the same manner as mentioned above.

In FIG. 3, 9 denotes a shielding member comprised of an opaque sheet or the like, which is disposed between the optical blocks. This shielding member 9 prevents, for example, a light ray that has been made incident on the optical block 3-5 from reaching imaging blocks other than the imaging block 5-5 corresponding to the optical block 3-5, that is, prevents light rays that have passed through the optical blocks from reaching imaging blocks other than the imaging blocks corresponding to the optical blocks, whereby occurring a so-called ghost is prevented.

Next, extraction of subject depth information by using the abovementioned compound eye imaging system 1 is explained. As mentioned above, in this embodiment, the optical axes of the optical blocks incline from each other so that the interval between the optical blocks become narrow at the subject side, and the optical axes roughly intersect each other at one point of intersection 11.

By such a construction, the imaging regions at the subject side in which each optical block picks up images and imaging regions in which other optical blocks pickup images overlap in the vicinity of the intersection 11.

In other words, images of a subject in the vicinity of the intersection 11 can be picked-up by separate imaging blocks through the plurality of optical blocks.

Each optical block is juxtaposed to other optical blocks, the optical blocks having pupils different from each other. Therefore, images of a subject with so-called parallax can be obtained.

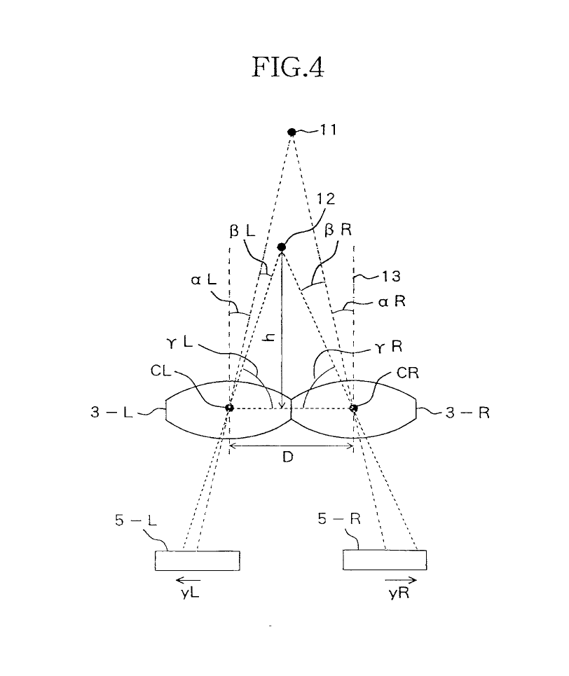

In FIG. 4, the compound eye imaging system 1 is partially described. In this figure, 3-L and 3-R show two optical blocks among the sixteen optical blocks. The optical axes of the optical blocks 3-L and 3-R intersect each other at the intersection 11.

Also, 5-L and 5-R shows imaging blocks corresponding to the optical blocks 3-L and 3-R. Among light rays from the subject, the image of a light ray that has passed through the optical block 3-L is formed on the imaging block 5-L, and likewise, the image of a light ray that has passed through the optical block 3-R is formed on the imaging block 5-R.

When the distance between the optical blocks 3-L and 3-R is defined as D, and parallel reference lines 13, shown by the alternate long and two-dashed lines, passing through the centers (CL, CR) at the principal point positions of the optical blocks are used, the angles αL and αR made by the reference lines 13 and the optical axes of the optical blocks and the distance D between the optical blocks are uniquely determined.

Herein, the object point 12 at the depth distance (distance to the object) h is examined. An image at the object point 12 is formed so as to shift from the centers of the imaging blocks by yL and yR on the imaging blocks 5-L and 5-R.

The focal lengths of the optical blocks 3-L and 3-R are known from the design values, so that the field angles βL and βR to the object point 12 from the optical blocks can be determined by yL and yR. Therefore, the base angles γL and γR of the triangles determined by the object point 12 and the points CL and CR are determined as follows:

γL=αL+βL

γR=αR+βR

Based on these, the depth distance h can be determined by means of trigonometry.

In this embodiment, the lens array 2 is comprised of a plurality of optical blocks. Therefore, to determine the subject depth distance, for example, not only the combination of the optical blocks 3-6 and 3-7, but also multiple pairs of optical blocks including, for example, a combination of optical blocks 3-5 and 3-7 and a combination of optical blocks 3-5 and 3-8 can be used.

By averaging the depth distances determined by using these multiple pairs of optical blocks, reliability on the measurement of the depth distance can be improved.

In the case where the depth distance is determined by means of trigonometry, generally, the accuracy in measurement of the depth distance increases as the reference line length, that is, the distance D between the optical blocks becomes longer. Therefore, the depth distance is measured by using a combination of the optical blocks 3-1 and 3-16 the distance D between which is the longest, whereby accuracy in measurement of the depth distance can be improved.

On the other hand, when the reference lines are taken to be long, in measurement of the distance to a subject with a three-dimensional shape, a measuring point at image pickup is possible by one optical block, however, it is impossible by another optical block since the point shaded by the subject, that is, a so-called occlusion occurs, and the measurement of the depth distance may become impossible.

In such a case, in this embodiment, for example, even when taking an image of a subject which causes occlusion by means of a combination of, for example, optical blocks 3-1 and 3-16, a combination of optical blocks the reference line lengths of which are shorter, for example, optical blocks 3-6 and 3-11 are used, whereby the measurement of the depth distance is possible.

The depth distance information thus measured is used for recognition of a person based on a distance difference in between the person as a subject and background to extract only the person image (with the background cut) and prevent the background from being picked-up in the case of, for example, surveillance camera systems and video teleconference systems.

In this embodiment, as shown in FIG. 3, the imaging element 4 can be driven in the direction of the arrow 13 by a drive means that is not shown. By driving the imaging element 4 with the drive means and adjusting the relative interval between the lens array 2 and the imaging element 4, for example, the image of a subject which is more distant than the intersection 11 can be formed on each imaging block through each optical block.

In this case, as mentioned above, since the optical axes of the optical blocks are disposed to incline from each other so that the optical axes of the optical blocks roughly intersect each other at the intersection 11 in this embodiment, when focusing a subject that is more distant than the intersection 11, different regions of the subject image can be picked up through the optical blocks. That is, the imaging region of the subject side is divided into a plurality (16 regions), and partial images of the subject can be obtained from the different imaging blocks for each divided region.

Therefore, by synthesizing the partial images outputted from the imaging blocks, an image of the entirety of the subject can be obtained.

In this embodiment, the optical blocks form inverted images of the subject on the imaging blocks. Therefore, to obtain an image of the entire subject from the outputs of the imaging block as an output from the imaging element 4, a signal processing circuit is required for converting the inverted images into erect images. It is desirable for reduction in the number of parts that this signal processing circuit is provided on an identical substrate to that of the imaging element 4.

In addition, in this embodiment, all the imaging blocks 5-1 through 5-16 are provided on the substrate of the imaging element 4. Therefore, when the imaging element 4 is driven by the drive means, all the imaging blocks 5-1 through 5-16 are collectively driven, so that adjustments of spaces between the optical blocks and imaging blocks become easier.

Furthermore, in this embodiment, the imaging element 4 is comprised of a single semiconductor substrate, and all the imaging blocks 5-1 through 5-16 are provided on the single semiconductor substrate. Such a plurality of imaging blocks are unified and provided on a single substrate, whereby the number of parts can be reduced, manufacturing of all the imaging blocks in the same manufacturing process becomes possible, and costs can be reduced.

Moreover, wiring inside the imaging element 4 can be formed when manufacturing the semiconductor substrate, so that the number of processes for assembly can be reduced.

Furthermore, the lens array 2 in this embodiment is manufactured by a press using a die or a mold, and all the optical blocks 3-1 through 3-16 are integrally formed. By such a construction, an assembly work for the lens array 2 and an adjusting work for the optical blocks can be omitted.

Furthermore, in this embodiment, the optical axes of the optical blocks incline from each other, and the optical axes of the optical blocks and the imaging blocks are not perpendicular to each other. In such a case, aberrations that are asymmetric with respect to the optical axes easily occur in the imaging blocks.

To prevent deterioration in the image quality due to such asymmetric aberrations, it is desirable that at least one of the optical action surfaces comprising the optical blocks is formed into an aspherical shape, and further formed into an aspherical shape which is rotational asymmetric to the optical axes.

It is also desirable for aberration correction that at least one of the optical action surfaces comprising the optical blocks is formed into a diffraction action surface. The use of a diffraction action surface is especially effective to correct chromatic aberrations.

In the abovementioned embodiment, the number of optical blocks is set to 16, however, the invention can also be applied to a compound eye imaging system with any number of optical blocks.

In the abovementioned embodiment, the case where imaging blocks corresponding to the optical blocks are provided on the imaging element is explained, however, a construction may be employed in which image detecting elements (pixels) are continuously, that is, correctively uniformly provided on the imaging element, and the imaging ranges corresponding to the optical blocks are set at different positions within these pixel groups.

(Second Embodiment)

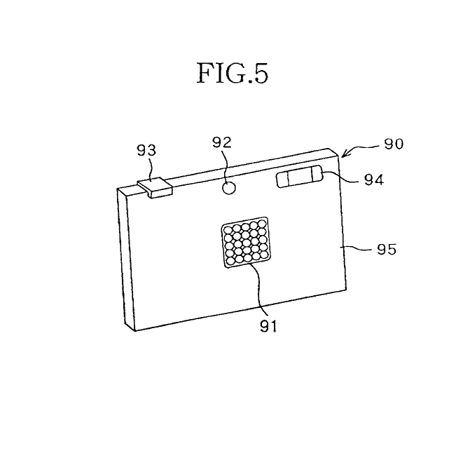

FIG. 5 shows a card type camera of a second embodiment of the invention. This camera 90 is comprised of compound eye imaging system 91 that is described in the first embodiment, finder window 92, shutter button 93, and flash 94 in the card type camera body 95.

An image that has been taken by the imaging element of the compound eye imaging system 91 is stored in an unillustrated memory in the camera body, and by inserting the camera 90 into a reader or the like connected to a computer that is not shown, the image can be displayed on the computer screen or printed-out.

(Third Embodiment)

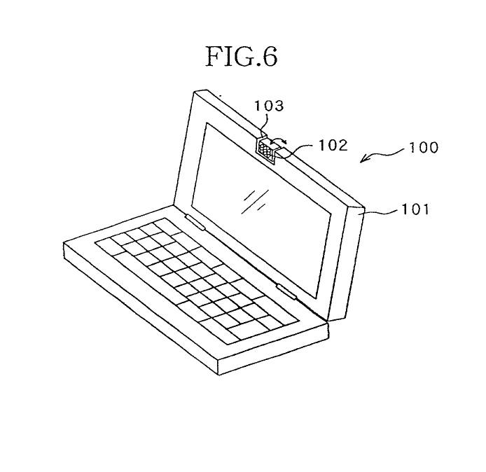

FIG. 6 shows a notebook or portable computer of a third embodiment of the invention. At the upper part of the screen 101 of this computer 100, an image taking part 103 with the compound eye imaging system 102 built-in as described in the first embodiment is rotatably held. The image taking part 103 is operated in accordance with the computer operation by a user, whereby a still image or moving image of the user or another subject can be taken.

As described above, according to the compound eye imaging system, since the optical axes of the optical blocks are made to intersect each other at the object side, at least one pair of object images (so-called, parallactic images) can be obtained through the optical blocks. Then, for example, based on the positional relationship of these object images on the imaging element and the focal lengths of the optical blocks, the distance to the object (depth distance) can be measured.

Also, if a drive means which relatively moves the compound eye optical system (lens array) and imaging element to change the space between the compound eye optical system and imaging element is provided, images of objects at varied distances can be formed on the image elements, and the distances to these objects can be measured.

Furthermore, when images of an object that is more distant than the intersection between the optical axes of the optical blocks are formed on the imaging element, images of the respective parts of the object are obtained through the optical blocks, and these partial images are synthesized, whereby an image of the entire object can be obtained.

By forming at least one of the optical action surfaces of each optical block into an aspherical shape, rotational asymmetric aspherical shape, or diffraction action surface, aberrations can be satisfactorily corrected.

What is claimed is:

1. A compound eye imaging system, comprising a plurality of optical blocks and an imaging element for picking-up object images formed by the optical blocks in imaging ranges provided for each optical block, wherein
optical axes of the optical blocks intersect each other at the object side.

2. A compound eye imaging system according to claim 1, wherein all the optical axes of the plurality of optical blocks roughly intersect each other at one point.

3. A compound eye imaging system according to claim 2, further comprising a drive means which relatively moves the plurality of optical blocks and the imaging element to change the space between the optical blocks and imaging element.

4. A compound eye imaging system according to claim 1, wherein a plurality of imaging blocks, which comprise the imaging ranges that are different from each other, are formed in the imaging element.

5. A compound eye imaging system according to claim 4, wherein the imaging element is constructed so that the plurality of imaging blocks are formed on a single substrate.

6. A compound eye imaging system according to claim 5, wherein the imaging element is constructed by forming the plurality of imaging blocks on a single semiconductor substrate.

7. A compound eye imaging system according to claim 1, wherein the plurality of optical blocks are unified.

8. A compound eye imaging system according to claim 1, wherein at least one of the optical action surfaces comprising the plurality of optical blocks has an aspherical shape.

9. A compound eye imaging system according to claim 8, wherein an optical action surface comprising at least one optical block of the plurality of optical blocks has a rotational asymmetric aspherical shape.

10. A compound eye imaging system according to claim 1, wherein at least one of the optical action surfaces comprising the plurality of optical blocks is a diffraction action surface.

11. An imaging device, comprising the compound eye imaging system according to claim 1.

12. An imaging device according to claim 11, wherein a distance to an object is measured based on outputs from a pair of imaging ranges for picking-up an image of the object through a pair of optical blocks in the imaging element.

13. An imaging device according to claim 11, wherein an average value of distances to an object is measured based on outputs from multiple pairs of imaging ranges for picking-up an image of the object through multiple pairs of optical blocks in the imaging element.

14. An imaging device according to claim 11, wherein an object image is synthesized by picking-up images of an object that is more distant than the intersection of the optical axes of the optical blocks by the imaging element.

15. An electronic equipment, comprising the imaging device according to any of claims 11 through 14.
